(19) United States
(12) Patent Application Publication
(10) Pub. No.: US 2005/0203586 A1
(43) Pub. Date: Sep. 15, 2005

(54) Electro-stimulating massage confiner

(76) Inventor: Tsung-I Yu, Taipei (TW)

Correspondence Address: BACON & THOMAS, PLLC, 625 SLATERS LANE, FOURTH FLOOR, ALEXANDRIA, VA, 22314, US

(21) Appl. No.: 10/791,658

(22) Filed: Mar. 12, 2004

(51) Int. Cl. A61N 1/18

(52) U.S. Cl. 607/46; 607/3

(57) ABSTRACT

An electro-stimulating massage confiner wholly or partially made by a flexible conducting fiber material is shaped to fit body portions, such as in a glove or a sock form. The massage confiner may consist of a single-polar conducting area or a pair or more than a pair of bipolar conducting areas. Each of the conducting area is provided with a conducting button for connecting a controller that can send out pulsating electric signals at low or middle frequencies, thereby urging an electro-stimulating massage effect over a selected body portion, such as a hand or a foot.

### BACKGROUND OF THE INVENTION

(a) Field of the Invention

The present invention relates to electro-stimulating massage devices, more particularly to an electro-stimulating massage confiner, wholly or partially woven by a conducting fiber material, taking forms of a glove or a sock for enclosing the corresponding body portions. Powered by a controller that outputs pulsating electric signals at low or middle frequencies, the electro-stimulating massage confiner provides a massage effect to the body portion it is mounted onto.

(b) Description of the Prior Art

The low-frequency electro-stimulating health assistors of the prior art commonly apply a set of electrodes, made of conducting silicon gel, attached onto human body portions such as hands, feet, neck, shoulders, etc. The assistors are connected to controllers that provide low-frequency pulsating electric signals for actuating an electro-stimulating massage effect to the body areas the electrodes cover. The conventional health assistors are disadvantageous in that the electrodes are easy to fall off the skin. To enhance the adhesive effect, sticky gel material is applied with the electrodes, bringing physical discomforts to a user.

The inventor of the present invention has disclosed some inventions about the electro-stimulating massage confiner using middle or low frequencies, such as those disclosed in U.S. patent Ser. Nos. 10/253,817, 10/352,135, 10/352,144, 10/403,126, 10/658,256, 10/658,188, 10/667,659, and 10/658,386, etc. In these inventions, middle or low frequency electro-stimulating massage devices are used to various portions of human body so as to achieve the object of massage.

### SUMMARY OF THE INVENTION

Accordingly, the primary objective of the present invention is to provide an electro-stimulating massage confiner that is wholly or partially made by a conducting fiber material and is shaped to fit body portions, such as in a glove or a sock form. The portion of the confiner made of conducting fibers may form a single-polar conducting area or a pair or more than a pair of bipolar conducting areas. Each of the conducting area is provided with a conducting button for connecting a controller that provides pulsating electric signals at low or middle frequencies. The controller causes a pulsating potential difference across a pair of conducting areas and thereby actuates an electro-stimulating massage effect over the body portion the conducting areas cover, such as a hand or a foot. Because of the excellent flexibility of conducting fibers, the confiner can attach a body portion so tightly that it would not fall off the body. Further, the electro-stimulating massage confiner is also easier and more comfortable to wear.

The various objects and advantages of the present invention will be more readily understood from the following detailed description when read in conjunction with the appended drawings.

### BRIEF DESCRIPTION OF THE DRAWINGS

FIG. 1 shows a preferred embodiment of the present invention as a glove of single electric polarity.

FIG. 2 shows a preferred embodiment of the present invention as a sock of single electric polarity.

FIG. 3 shows a preferred embodiment of the present invention as a glove having two opposite electric polarities.

FIG. 4 shows a preferred embodiment of the present invention as a suck having two opposite electric polarities.

FIG. 5 shows the preferred embodiment of a single-polar glove connected to a controller.

FIG. 6 shows the preferred embodiment of a single-polar suck connected to a controller.

FIG. 7 shows a preferred embodiment of the present invention as a bipolar glove connected to a controller by a wire.

FIG. 8 shows a preferred embodiment of the present invention as a bipolar glove having a controller directly mounted thereon.

FIG. 9 shows a preferred embodiment of the present invention as a bipolar suck with a separate controller.

FIG. 10 shows a preferred embodiment of the present invention as a bipolar suck with a controller attached thereon.

FIGS. 10 to 14 are bottom views of the present invention as a sock having various arrangements of conducting areas.

FIG. 15 shows a preferred embodiment of the present invention having a plurality of bipolar conducting sets and the attached conducting buttons.

FIG. 16 shows a preferred embodiment of the present invention as a sock having a plurality of bipolar conducting sets corresponding to the acupuncture points on the bottom side of a foot.

FIG. 17 shows a preferred embodiment of the present invention as pantyhose having a bipolar conducting set.

FIG. 18 is an exploded perspective view of the present invention as an insulating mitten including a pair of conducting clothes of opposite polarities.

FIG. 19 is a cross-sectional view of the present invention as the mitten in FIG. 18.

FIG. 20 is a perspective view of the present invention as a bipolar ring object.

FIG. 21 is a perspective view of the present invention as a single-polar sleeve.

FIG. 22 is a perspective view of the present invention as a bipolar sleeve.

FIGS. 23 to 26 show the application of the present invention as a bipolar sleeve to various portions of a human body.

### DETAILED DESCRIPTION OF THE PREFERRED EMBODIMENTS

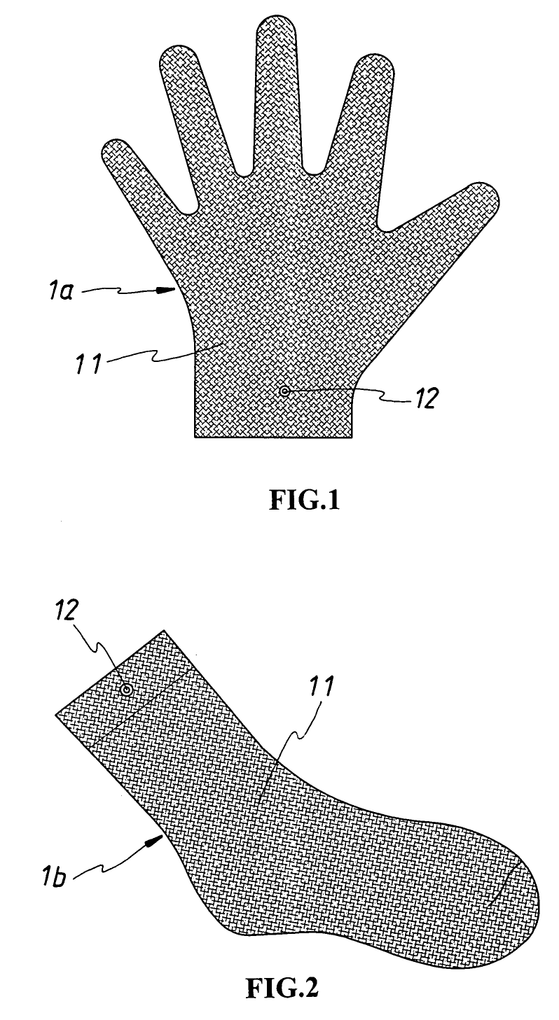

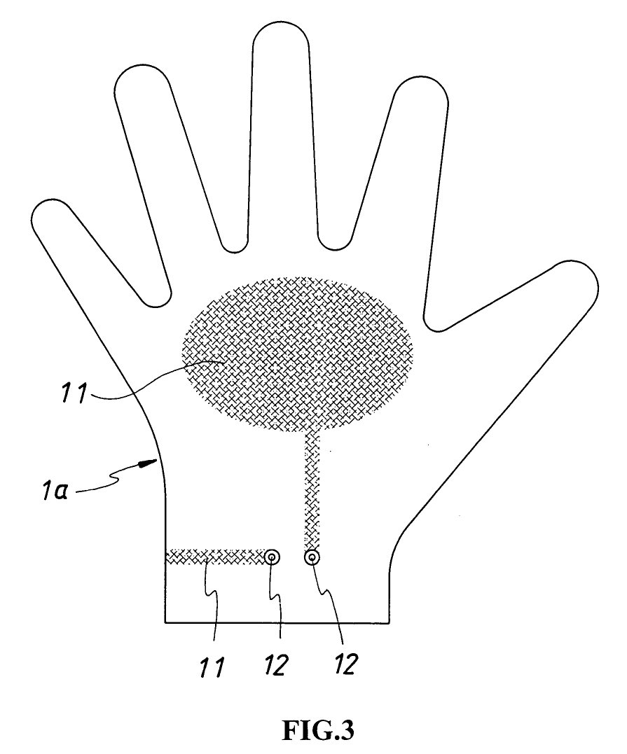

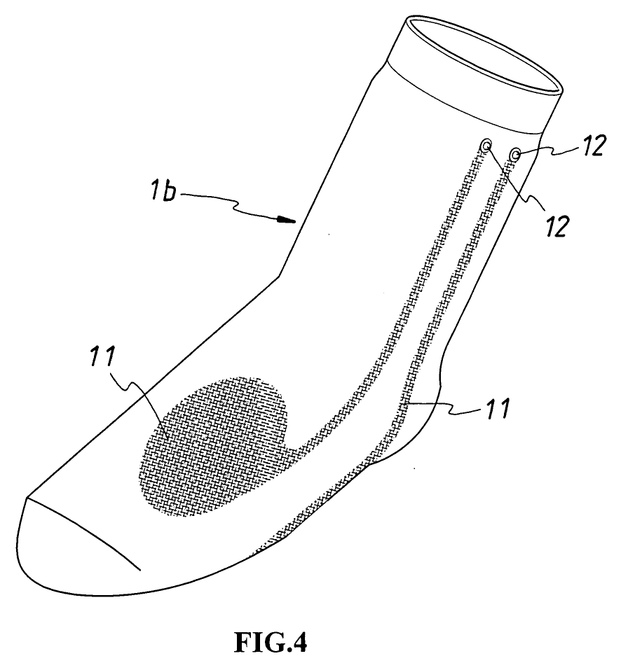

Referring to the appended drawings, the present invention as an electro-stimulating massage confiner is wholly or partially woven by flexible conducting fibers and may take various forms, such as a glove 1a (as shown in FIGS. 1 and 3), a sock 1b (as shown in FIGS. 2 and 4) or other shapes fitting a body portion. The massage confiner, in form of a glove 1a or a sock 1b, can be provided with a single polar conducting area or a pair or more than a pair of conducting areas of opposite polarities (11); each conducting area is further provided with a conducting button 12. Thereby, the glove 1a or the sock 1b can be connected to a controller 2 that can output pulsating electric signals so as to provide an electro-stimulating massage effect to a corresponding body portion to which it is attached.

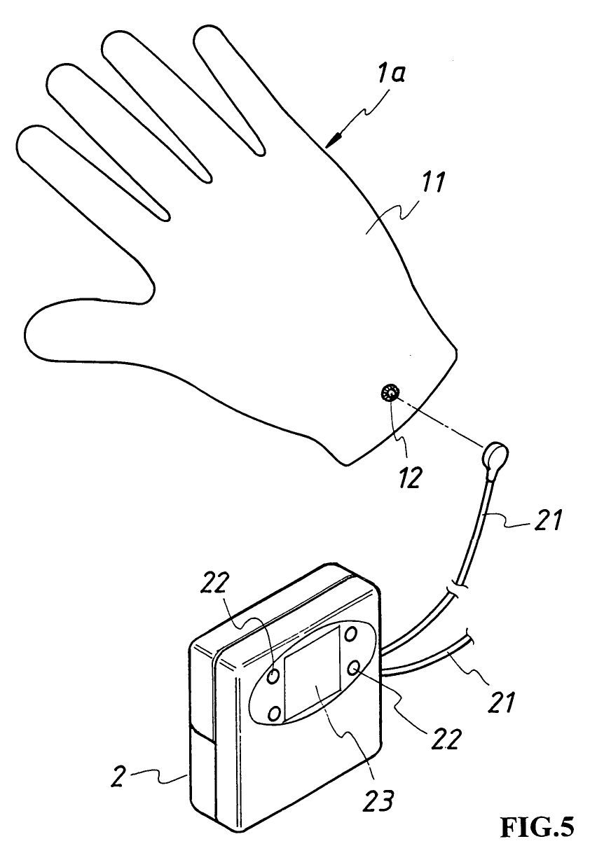

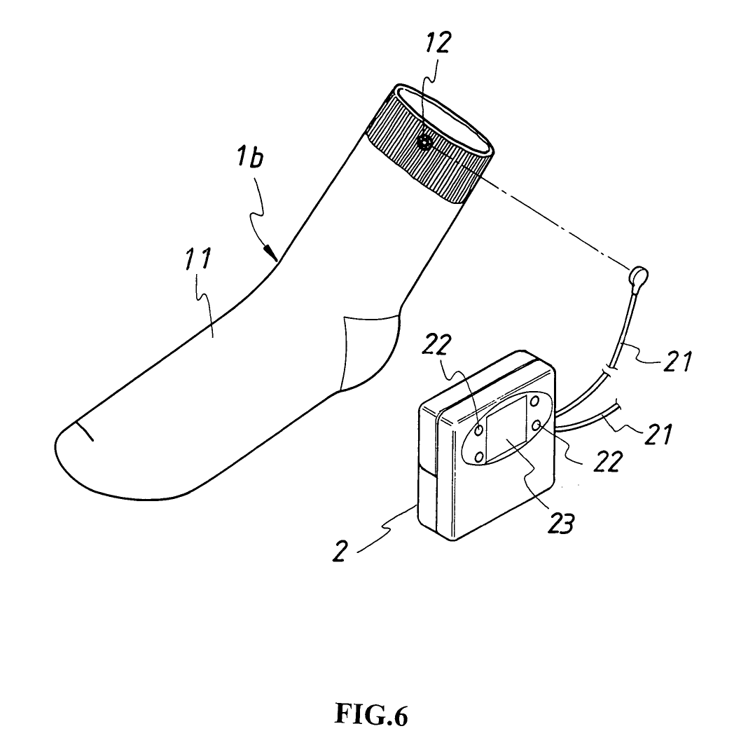

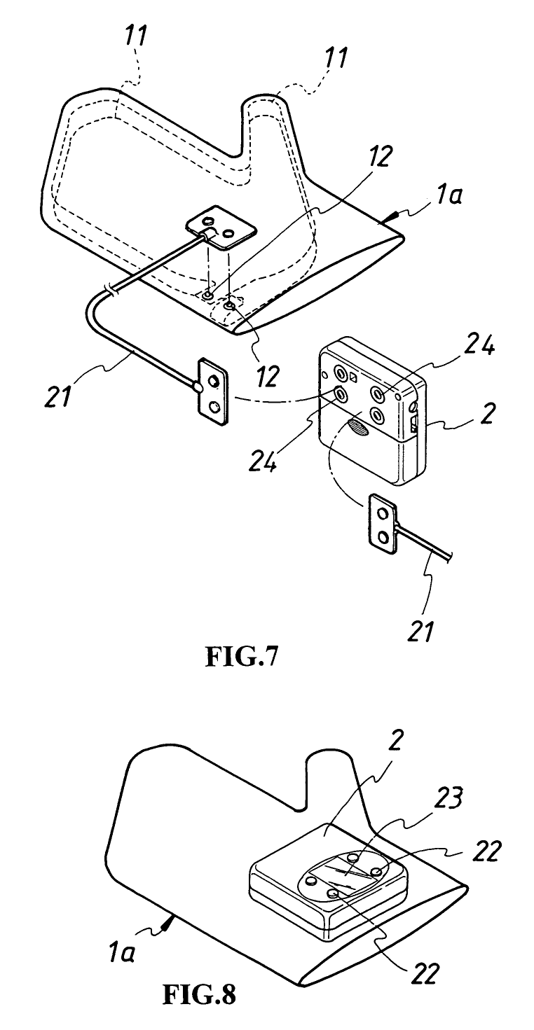

As shown in FIGS. 5 and 6, the foresaid single polar massage confiner 1a or 1b is connected to a controller 2 by means of connecting a conducting button 12 with the controller 2 using a wire 21, so as to form a closed loop. A bipolar massage confiner 1a, as shown in FIG. 7, is connected to a controller 2 by means of connecting two conducting buttons 12 of opposite polarities with the controller 2 using a wire 21. Thereby, the massage confiner 1a can be used alone to provide an electro-stimulating massage effect.

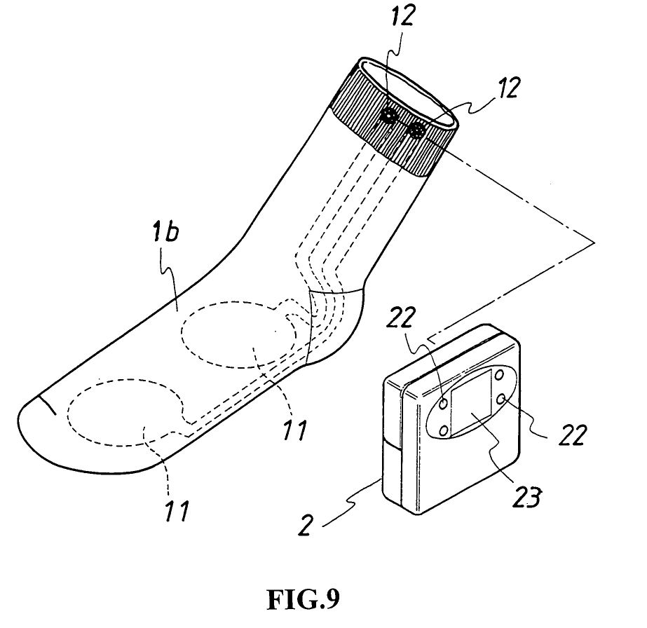

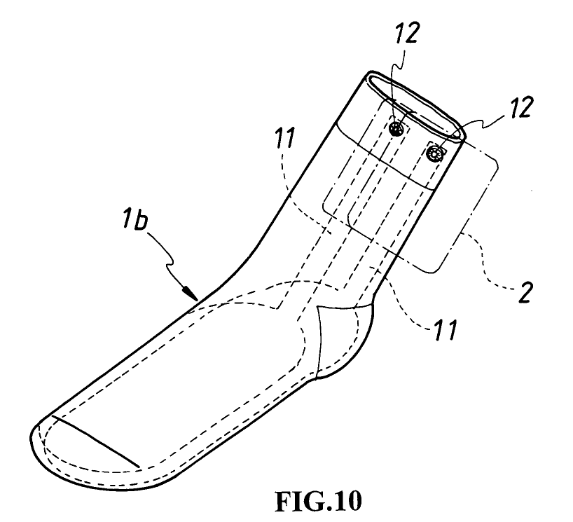

As shown in FIGS. 8 to 10, a bipolar electro-stimulating massage confiner, embodied in 1a or 1b, can be directly coupled with a controller 2, so that a wire can be omitted.

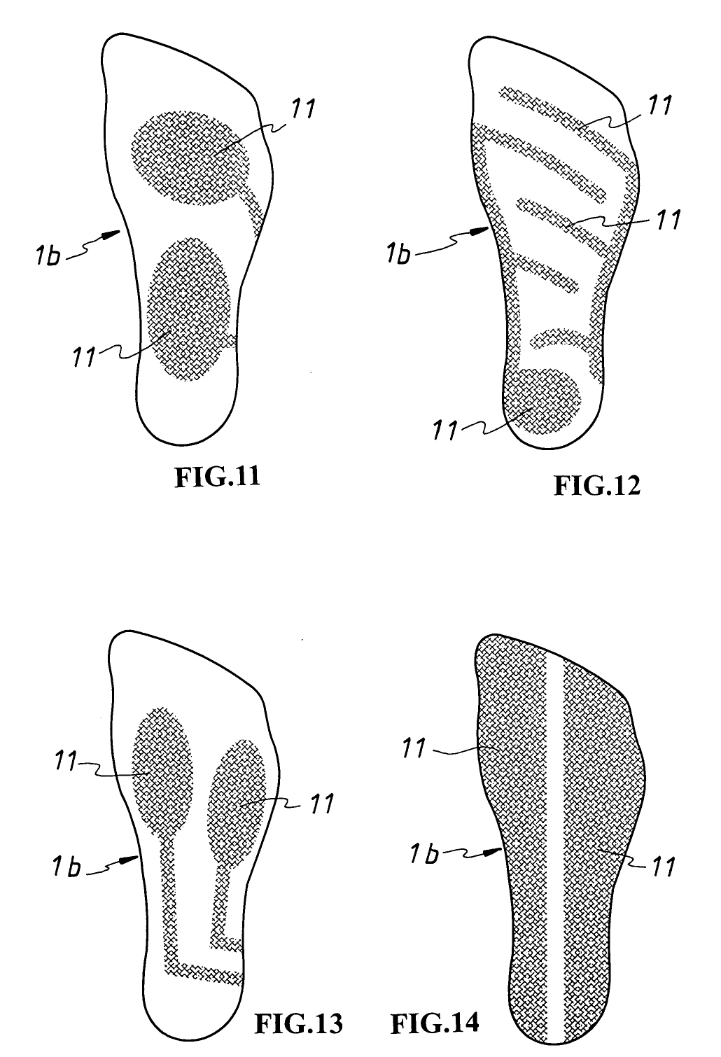

According to the spirit of the present invention, the massage confiner, as a glove 1a or as a sock 1b, may have a plurality of conducting areas 11 arranged thereon according to a selected distribution. If the arrangement is such that the conducting areas 11 are aligned with the acupuncture points over a body area, the pulsating signals can provide an electro-stimulating massage effect to the acupuncture points. As shown in FIGS. 11 to 14, the present invention embodied in a sock 1b may have various patterns of conducting area distribution. By the same token, the present invention embodied in a glove 1b may have various patterns.

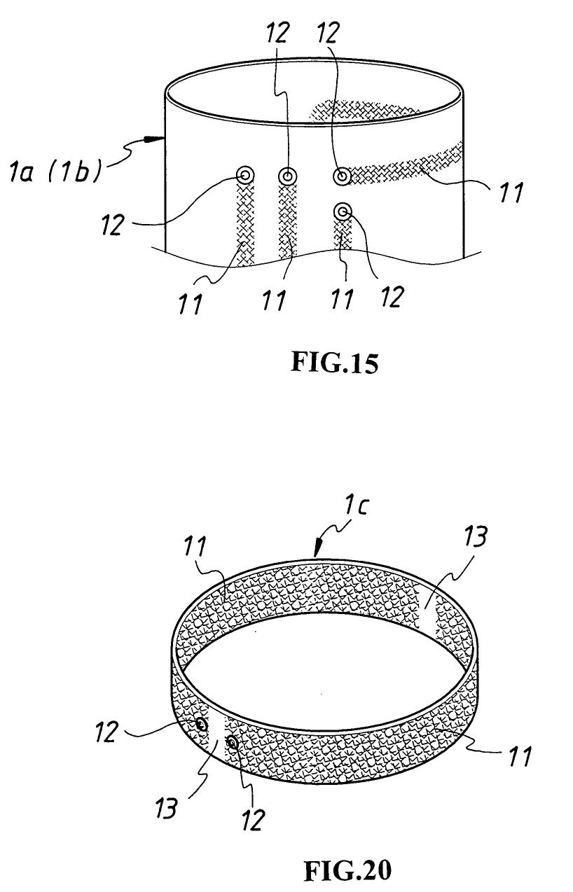

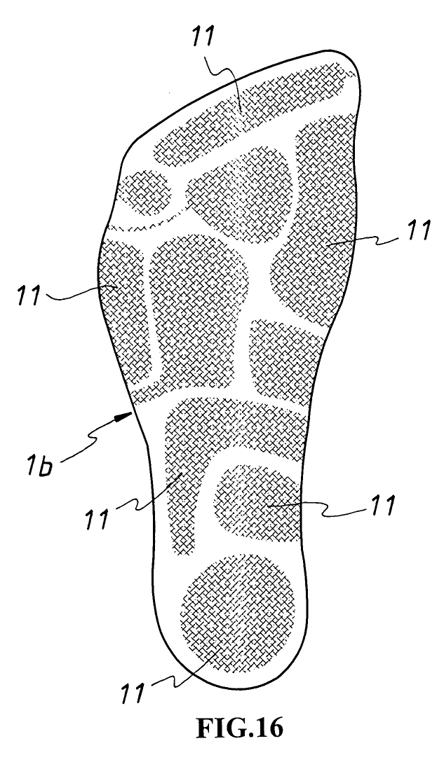

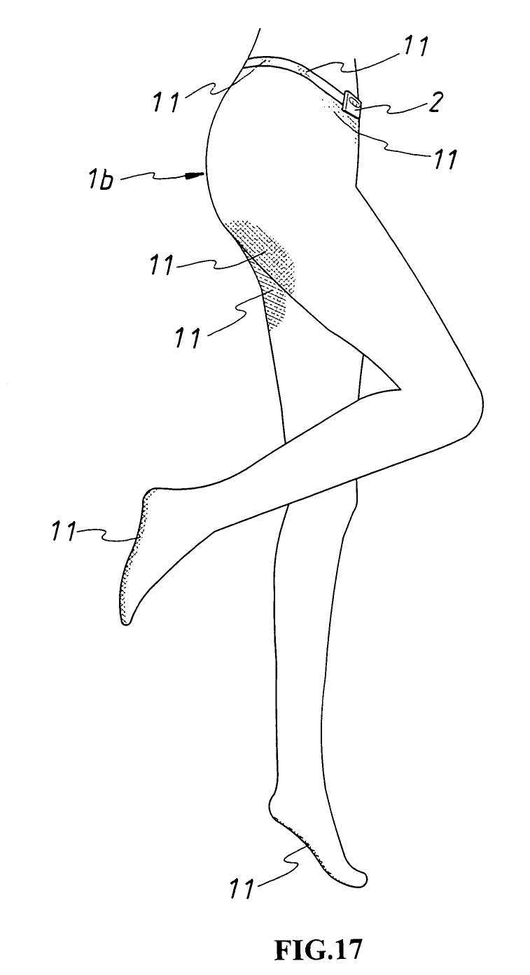

The foresaid massage confiner having a plurality of conducting areas 11, as a glove 1a or as a sock 1b, are further provided with conducting buttons 12, each for a conducting area 11, as shown in FIG. 15. Thereby, a controller 2 may connect a selected pair of conducting buttons 12 so as to provide an electro-stimulating massage effect to a selected acupuncture point on a foot or a hand. As shown in FIG. 16, the present invention embodied in a sock 1b has a plurality of conducting areas 11, woven by conducting fibers and arranged in interlaced polarity distribution according to a selected pattern. When the sock 1b is worn on a foot, various acupuncture points on the foot can be massaged. By the same token, the present invention can be embodied in a pantyhose 1b, as shown in FIG. 17. The pantyhose 1b may have a single polar conducting area of a pair or more than a pair of conducting areas 11 and the corresponding conducting buttons 12.

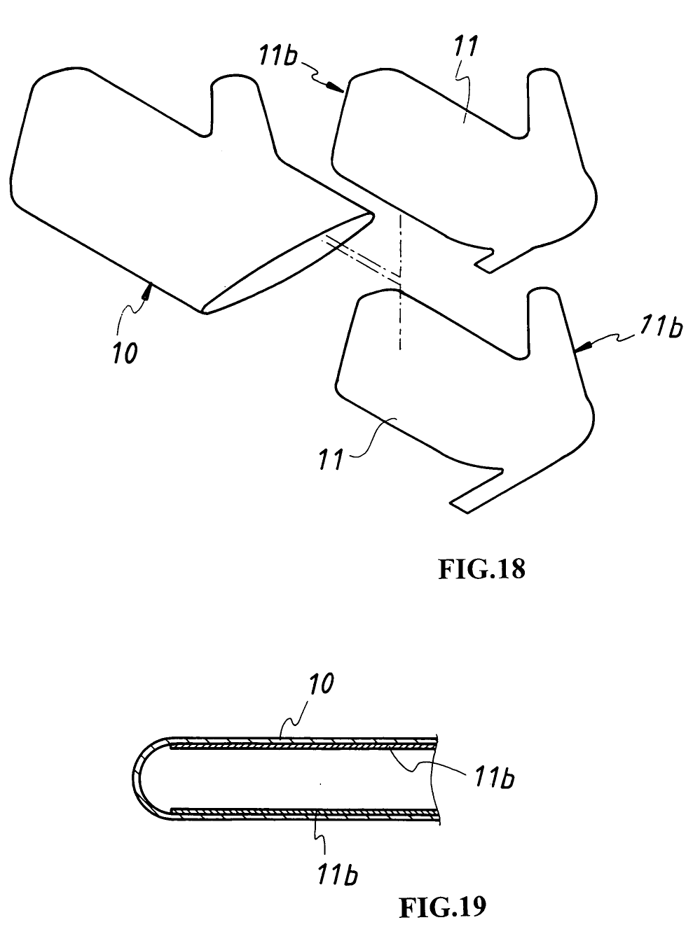

As shown in FIGS. 18 and 19, the present invention can also be embodied in two conducting cloths 11b each made of conducting fibers. Two conducting cloths 11b are respectively attached onto the upper inner surface and the lower inner surface of an insulating mitten 10 to form conducting areas 11 of opposite polarities. The mitten 10 thus formed can provide an electro-stimulating massage effect across the hand inserted therein. The mitten 10 is further provided with conducting buttons 12 at selected locations for connecting a controller 2.

The various embodiments of the present invention are thus described, and it will be obvious that the same may be varied in many ways. Such variations are not to be regarded as a departure from the spirit and scope of the present invention. As shown in FIG. 20, the electro-stimulating massage confiner of the present invention can be a ring sleeve 1c for encircling a body portion. The ring sleeve 1c may have a single polar conducting area 11 or a pair or more than a pair of conducting areas 11 and conducting buttons 12 each for a conducting area 11, so that a controller 2 transmitting pulsating electric signals at low or middle frequencies can be connected. The controller 2 causes a pulsating potential difference across two conducting areas 11 of opposite polarities to provide an electro-stimulating massage effect to the applied body area. Since the ring sleeve 1c in FIG. 20 consists two conducting areas 11, an insulating section 13 made of a flexible insulating material, such as a flexible band, is necessary.

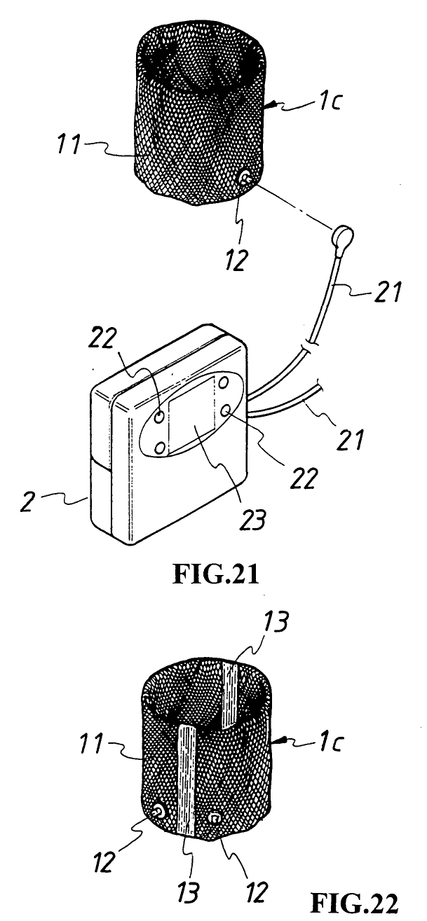

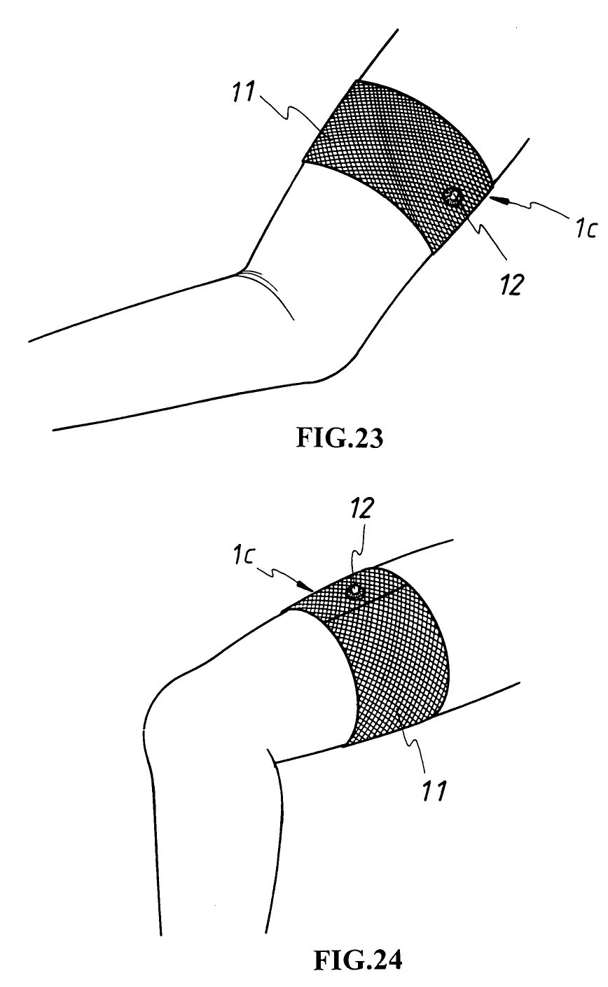

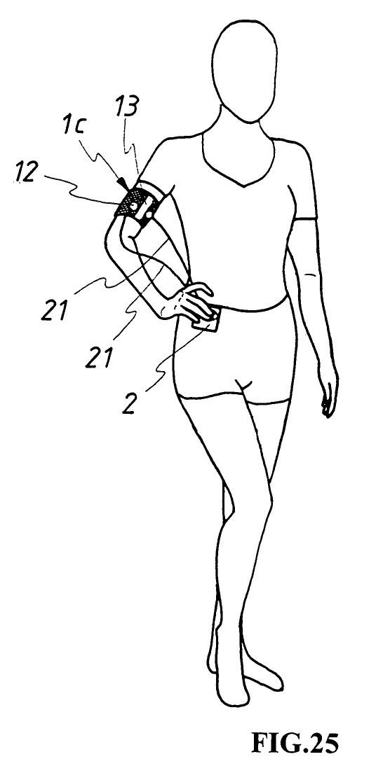

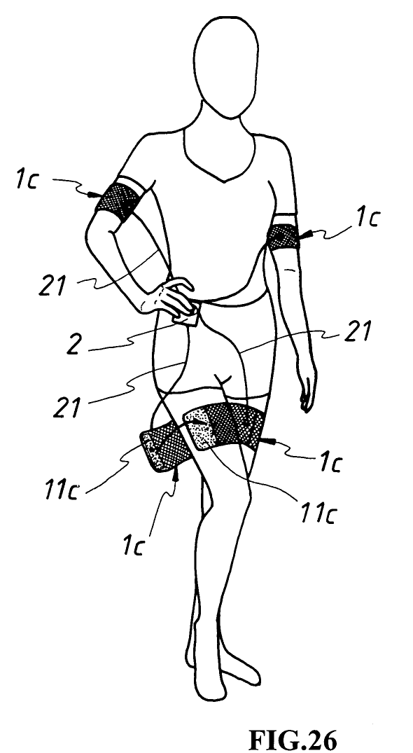

The present invention as an electro-stimulating massage confiner can also form a flexible barrel sleeve 1c, as shown in FIGS. 21 and 22. FIGS. 23 to 26 illustrate the flexible barrel sleeve 1c, woven in a net pattern, being applied to various portions of a human body. The barrel sleeve 1c can further include an adhesive member for tightening the barrel sleeve 1c to a body portion.

The aforesaid controller 2 includes a central integrated circuit (IC) for transmitting pulsating electric signals so as to produce high-voltage output by repeatedly charging and discharging capacitors and inductors therein. The pulse width of the output signals is program-controlled, ranging from 1 Hz to 150 Hz, so that massage of various strengths can be produced. The controller 2 is provided with a plurality of control buttons 22 for respectively adjusting the up and down of electric current, massage time, power on/off and massage modes. It further includes an LCD panel 23 for displaying the control status. The high-voltage pulsating output from the internal circuit in the controller 2 is connected to a plurality of conducting buttons 24 (FIG. 7) to form an output terminal, which can be connected to corresponding conducting buttons 12 on a massage confiner, a glove 1a, a sock 1b or other forms 1c, through wires 21 so as to form a closed loop.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. An electro-stimulating massage confiner, comprising:
an enclosure for fitting a portion of human body having a plurality of conducting areas made of a conducting fiber material, said conducting areas having an arrangement selected from the group of forming an integral area of a single electric polarity, a pair of areas of opposite polarities and a plurality of pairs of areas of opposite polarities, said conducting areas having a coverage selected from the group of whole said enclosure and partial regions of said enclosure, said conducting areas being separated by an insulating material; and
a plurality of conducting buttons each corresponding to one said conducting area;
whereby said conducting buttons of said electro-stimulating massage confiner can be connected to a controller so as to produce an electro-stimulating massage effect.

2. The electro-stimulating massage confiner of claim 1 wherein said conducting areas are a plurality of areas having an extended distribution over said massage confiner.

3. The electro-stimulating massage confiner of claim 1 wherein said enclosure is provided with a plurality of pairs of conducting areas having opposite electric polarities, and wherein each of said conducting areas has a corresponding conducting button.

4. The electro-stimulating massage confiner of claim 1 wherein said enclosure is a sock and a plurality of said pairs of conducting areas of opposite electric polarities are disposed at selected locations on a bottom side of said sock.

5. The electro-stimulating massage confiner of claim 4 wherein said pairs of conducting areas are distributed corresponding to the acupuncture points on the bottom side of a foot.

6. The electro-stimulating massage confiner of claim 1 wherein said enclosure is a glove and said conducting areas of opposite electric polarities are respectively attached onto an upper inner surface and a lower inner surface of said glove.

7. The electro-stimulating massage confiner of claim 1 wherein said enclosure is a ring sleeve composed of two conducting areas of opposite electric polarities and insulating sections that divide said conducting areas.

8. The electro-stimulating massage confiner of claim 7 wherein said insulating sections are made of a non-conducting, flexible material.

9. The electro-stimulating massage confiner of claim 1 wherein said enclosure is a ring sleeve formed by enclosing a band by a plurality of adhesive members thereon.

10. The electro-stimulating massage confiner of claim 1 wherein said controller includes a central integrated circuit for transmitting pulsating electric signals that charges and discharge capacitors and inductors therein so as to generate high-voltage pulses; and wherein the pulse width of said high-voltage pulses is program-controllable for providing a massage effect of various strengths at low and middle frequencies.

11. The electro-stimulating massage confiner of claim 13 wherein said pulse width of said electric pulses produced by said controller ranges from 1 Hz to 150 Hz

12. The electro-stimulating massage confiner of claim 10 wherein a sequence of high-voltage pulses produced by said controller provides are sent to a plurality of conducting buttons to form an output terminal.
